(19) United States
(12) Patent Application Publication
(10) Pub. No.: US 2023/0009133 A1
(43) Pub. Date: Jan. 12, 2023

(54) SOFT TISSUE EXPANDER FOR CLEFT LIP AND PALATE REPAIR

(71) Applicant: Regents of the University of Minnesota, Minneapolis, MN (US)

(72) Inventors: David Nedrelow, Minneapolis, MN (US); Ishaan Duggal, Austin, TX (US)

(21) Appl. No.: 17/859,645

(22) Filed: Jul. 7, 2022

Related U.S. Application Data
(60) Provisional application No. 63/203,066, filed on Jul. 7, 2021.

(51) Int. Cl. A61B 90/00; A61L 31/04; A61L 31/10; A61L 31/14; G06F 30/17

(52) U.S. Cl. CPC A61B 90/02; A61L 31/041; A61L 31/10; A61L 31/148; G06F 30/17; A61B 2017/00792

(57) ABSTRACT

Treatment of palatal defects is accomplished through devices that have controlled start time. The devices expand directionally to provide appropriate levels of stress and strain to a target tissue to promote tissue growth over the course of days or weeks, even as the defect repairs itself.

### PRIORITY CLAIM

The present application claims priority to U.S. Provisional Application Ser. No. 63/203,066 filed Jul. 7, 2022, and entitled “SOFT TISSUE EXPANDER FOR CLEFT LIP AND PALATE REPAIR”, which is hereby incorporated by reference in its entirety.

### TECHNICAL FIELD

Embodiments described herein relate to treatment of cleft palate or similar conditions for which stimulated growth of mucosal tissue is advantageous through the use of applied oral mucosa expansion.

### BACKGROUND

Cleft lip and palate is one of the most common congenital anomalies in children. Despite advances in local flap graft and free flap graft surgical techniques, complications such as fistula formation, or a need for pharyngoplasty can occur after cleft closure. For difficult cleft palate repair cases, many revision surgeries can be necessary numbering in the dozens or more. It is thought that wound contractures may be an important factor in the following cleft palate repair morbidities: (1) soft palate hypoplasia, (2) conductive hearing loss, difficulties with (3) feeding and (4) speech, and (5) restricted growth in gross skeletal structures of the head and face.

Soft tissue expanders have been widely used for the purposes of reconstructive surgery. New skin is grown adjacent to the surgical site through the use of an implantable expander device which stimulates skin growth by applying a sustained stretch. The new skin is useful from a surgical perspective because it expands the area that can be covered by local flap grafts.

A wide range of repairs have been made with expanders such as repairs to birth defects or burns, and for breast reconstruction. For example, U.S. Pat. No. 6,228,116 describes a tissue expander based on a gas-containing member and multiple chambers. Non-gas-based devices are also known for other treatments, such as WO 2015/160699, describing the use of crosslinked hydrogels impregnated with a drug. Self-inflating devices are also known, such as WO 2007/080391, which is based on a polyvinylpyrrolidone (PVP)/acrylate copolymer.

While these solutions are useful in healing or reconstructing tissue, they can require multiple operations to adjust the mechanics of expansion.

### SUMMARY

According to an embodiment, a device for palatal repair includes a plurality of polymeric fibers arranged into a body, the body configured to expand directionally upon exposure to a liquid, and a coating configured to provide a controlled start time of the device.

According to another embodiment, a method for designing a palatal repair device includes capturing a three-dimensional image of a palatal defect with tissues adjacent to the palatal defect; selecting an anchor location for the palatal repair device; specifying an initial device geometry that is compatible with an anatomy adjacent the palatal defect; determining an appropriate material or set of materials and corresponding arrangement within the device to generate tissue growth; modeling implanted device expansion, expected strain and stress at the palatal defect; and expected growth or resorption of soft tissue and bone, wherein a growth index is computed that describes soft and hard tissue growth; and modifying the anchor location, device geometry, or arrangement of self-inflating materials within the device based upon the model, and repeating iteratively to improve palatal defect repair speed and reduce tissue deficits.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 shows isometric, top, and side views of a device according to a first embodiment, in the unexpanded state.

FIG. 2 shows the device of FIG. 1 in an expanded state.

FIG. 3 shows isometric, top, and side views of another device according to a second embodiment, in the unexpanded state.

FIG. 4 shows the device of FIG. 2 in an expanded state.

FIG. 5 is a flowchart depicting a method for determining a device design, according to an embodiment.

FIG. 6 is a simplified model of a bone-mucosal tissue interface with an expander therebetween.

FIGS. 7A-7I depict a system for generating mucosal tissue growth according to a dome-shaped embodiment of an expander device.

FIGS. 8A-8I depict a system for generating mucosal tissue growth according to a rectangular-shaped embodiment of an expander device.

FIGS. 9A-9I depict a system for generating mucosal tissue growth according to a cone-shaped embodiment of an expander device.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments described herein include novel devices and treatment methods that provide cleft repair in a matter of weeks, without requiring numerous follow-up medical procedures required by conventional treatments. Embodiments described herein promote six important features of a cleft repair device: (1) the devices are self-initiating; (2) the devices have directional expansion characteristics; (3) the devices are biocompatible; (4) the devices have controlled start; (5) the devices are “4D-printed” such that they can be (A) fabricated using a 3D-printer, and (B) grow in each of the three spatial directions as desired over time; and (6) the devices are both mucosa- and bone-growing. These concepts are described in more detail below, in relation to embodiments that provide all of the desired attributes so that cleft palates can be repaired without multiple operations.

As described herein, these factors are all considered in establishing a biomechanics framework to cleft palate birth defects by applying both computer modeling and device design principles. The proposed devices and methods aim to leverage mechanotransduction concepts to engineer new palatal mucosa, thus reducing wound contracture and the accompanying morbidities described above that are associated with cleft palate repair.

Devices and methods described herein rely on the body's growth reaction to applied mechanical stress. A cleft palate or other defect can be analyzed using finite analysis or other techniques to estimate a level of stress or strain based on the size and shape of an implant. Stress relaxation can be measured or estimated, and separated into two bins: one bin of data before the peak stress and one bin after the peak stress. Using such techniques, the relaxation time of the tissues can be determined (or estimated) for bone, cartilage, and soft tissue of a cleft palate or other defect that is being treated.

Using such information, a soft-tissue expander can be designed and generated for implant in or nearby to the defect to generate a desired level of stress or strain that causes tissue growth in a desired rate, region, or type. An understanding of how the strain and stress field propagates throughout the cleft or other defect is important to understanding how soft tissue growth can occur as a result of overstretch, for example.

The areas of high mucosal strain in or around a cleft palate are likely to exhibit growth when the strain is sustained over time. In theory, the additional mucosal growth generates more free tissue for local flap closure of the palatal defect which would allow palate closure without a free flap, or with less tension to the wound site and thus improved patient outcomes.

The primary expander technologies that have been considered for cleft palate repair are: (1) balloon expanders, and (2) osmotic expanders (Shash et al. 2015). Some success was met with the former, but balloon expanders require a port into which saline injections must be repeatedly administered throughout the expansion, and thus remain a feasibility challenge to widespread adaptation (Mey et al. 1990; Van Damme and Freihofer 1996). The latter self-inflating expanders were later used to enhance cleft palate repair and overcome challenges with balloon expanders (Kobus 2007). But, the preliminary results suggested that without the careful application of forces, osmotic expanders caused pathologic overloading in soft and hard tissues. More recently, (Swan et al. 2012) formulated a new material with directional control over expansion, but again, overloading of the tissues resulted in the formation of large bathtub depressions in palatal bone.

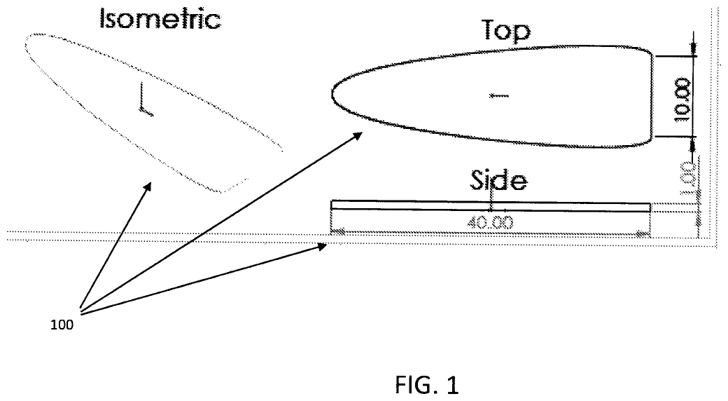

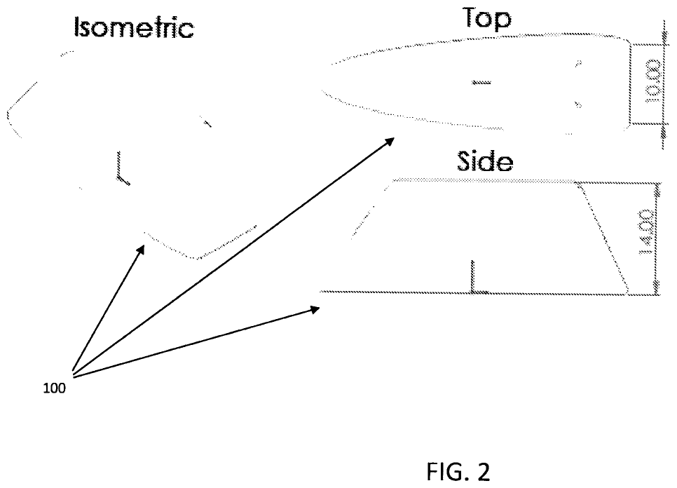

Devices described herein do not rely on such high levels of mechanical stress on the palatal defect. One example of an improved device is shown in FIGS. 1 and 2. In FIG. 1, device 100 is in an insert 100 usable for palatal defect repair. In use, device 100 would be implanted at or nearby the palatal defect. The shape and size of insert 100 can be designed as described below to correspond to the shape and size of the defect, as well as its expected shape and size over time. FIG. 2 shows the same device 100, but in an expanded state.

Self-Inflating

Devices described herein have a property referred to as self-inflating. Self-inflating, or swelling, refers to growth over time without external manipulation. Inflation does not necessarily refer to filling the center of the device 100 with a gas or a liquid. Rather, self-inflating refers to the device plumping over a period of time. This is helpful in treatment of palatal defects because as the device 100 self-inflates, it can be used to modify the strain applied over time (see the section entitled “4D-Printed,” below).

Self-inflation or swelling is caused by both the choice of materials used in the device and thermomechanical preconditioning of the material. A combined mixture of the following polymers in various ratios into a copolymer can provide control over the rate of expansion. Materials that may be used to form device 100 can include, but are not limited to:
Methylcellulose
Poly(acrylamide)
Poly(caprolactone)
Poly(ethylene glycol)
Poly(glycolic acid)
Poly(glycerol sebacate)
Poly(2-hydroxyethyl methacrylate)
Poly(lactic acid)
Poly(propylene fumerate)
Poly(vinyl alcohol)
Poly(vinyl pyrrolidone)
Poly(methyl methacrylate)

Materials can be selected to have different molecular weights which will affect their mechanical properties. For example, higher molecular weight generally causes an increase in stiffness. In one embodiment, device 100 can comprise a Poly (vinyl alcohol)-graft polyurethane with resorcinol. The Poly (vinyl alcohol)-graft polyurethane with resorcinol can be originally made as a pva-graft-pcl, then reacted with diisocyanate and resorcinol for use in forming and tuning device 100.

After a primary form is made from the material, a secondary shape can be programmed through the use of thermomechanical conditioning. Cycles of heating and cooling can be applied to the device to introduce anisotropies. For example, the appropriate heating is applied after compressing the material, followed by cooling and releasing pressure, will cause the material to assume a secondary insertion configuration. Then, after implantation, the material returns to its primary shape.

Swelling starts when the material that makes up the body of device 100 comes into contact with the interstitial fluid after implantation. Device 100 can be coated such that self-inflation does not begin until the desired time, even if device 100 is in humid environments or is briefly exposed to water, for example.

Directional Expansion

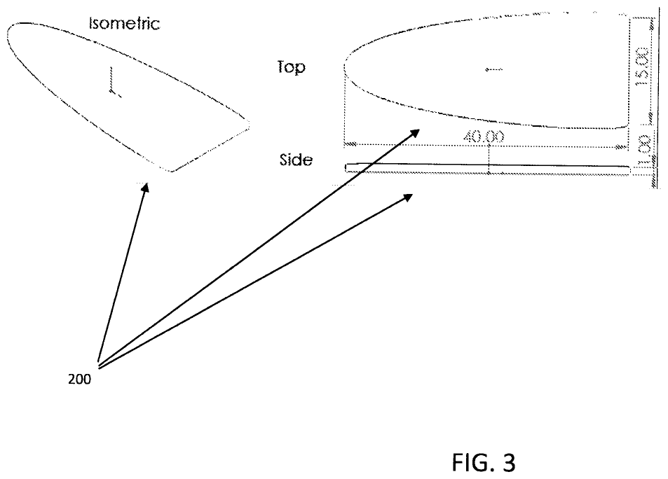

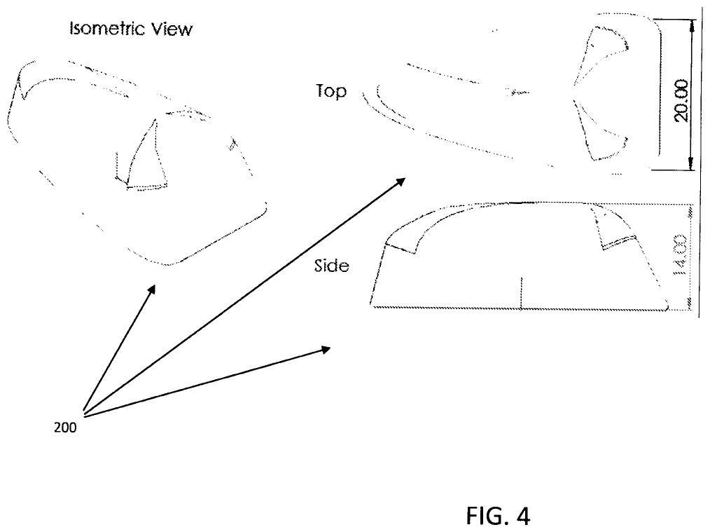

As shown in the difference between FIGS. 1 and 2, the initial shape of the device 100 affects its final size and shape after swelling. Choice of material affects both rigidity and the total size increase when self-inflation occurs. In the embodiment shown in FIGS. 1 and 2, the top view of device 100 is largely unchanged, while the side view is significantly different, due to the materials and shape selected for that device 100. In alternative embodiments (such as the embodiment shown in FIGS. 3 and 4, below) the materials and initial shape can be selected to cause a different type of directional expansion altogether.

Directional expansion is important because it facilitates the repair of palatal defects having specific shapes, and gives a medical professional more freedom in determining where to place the device. Furthermore, directional expansion can be designed such that multiple dimensional expansions occur independent from one another which is important for preventing unwanted device extrusion. For example, the lateral width of the device can be made to expand after implantation such that tissues adjacent to the insertion site provide mechanical retention.

Biocompatible

As described above, the materials that make up device 100 are typically biocompatible polymers, so that the patient will not adversely react to the device being implanted for a period of time that can range from days to several weeks before removal. When a coating is used, that coating can also be selected from biocompatible materials that are unlikely to cause adverse reactions when implanted into a patient's mucosa. For example, coatings could be selected from:
Poly(ethylene glycol)
Silicones
Poly(ethylene glycol) diacrylate

Other materials that may be used, such as initiators, plasticizers, fillers, and crosslinkers can also be selected from biocompatible materials.

Controlled Start

As described above, coatings can be used that set the start of the swelling or self-inflation process, which proceeds in a directional manner. Coatings can prevent inadvertent contact with water or other fluids from initiating the growth of device 100. For example, a coating could be designed to break down over a period of minutes, hours, or days in contact with interstitial fluid such that the body material that makes up the bulk of device 100 is not exposed until after the device is inserted.

Furthermore, the area around incision of the device 100 can be sensitive or susceptible to damage during healing after insert the device 100. If desired, a coating could be implemented to delay the beginning of growth of the main body material of device 100 for longer, such as several hours to a few days.

4D-Printed

Device 100 is “4D-Printed,” which is a term used herein to refer to a design that takes into account not just the shape and size of the initial palatal defect, but the expected size and shape of the palatal defect over time as it heals and fills in due to the mechanical stress applied by the device 100. By modeling the stress and strain on the palatal defect in advance, the growth rate, desired size, and desired shape of the device 100 can be determined. In this way, the device 100 can be printed from one or more materials having expansion characteristics that will result in continued application of mechanical stress and strain on the palatal defect over a course that can last for between about 1 and 60 days in a typical regimen. During this entire time period, the device 100 can apply sustained pressure to a tissue area such that an elastic area stretch between 1.1 and 1.5 (and the corresponding stress) is achieved.

By 4D-printing the device and modeling its expected applied force over the entire regimen, design of device 100 is tunable to ensure that the stress does not become less than, or greater than, the desired pressure amount. One way that the device can be tailored to achieve the desired pressure amount is by tuning the device to have a desired elastic area stretch ratio (elastic area stretch ratio is equal to the stretched area (length times width) divided by the unstretched area (original length times original width)). Forces that result in elastic area stretch ratios significantly less than 1.1 are unlikely to cause significant soft tissue growth, while elastic area stretch ratios significantly greater than 2 can cause discomfort or injury to the patient.

FIGS. 3 and 4 show an alternative device 200 that begins in an unexpanded state (shown in FIG. 3) that is quite similar to device 100 in the unexpanded state (shown in FIG. 1). Device 200 has been tuned, however, by modifying the specific materials arranged in the body thereof to create a lobed expanded shape (shown in FIG. 4). As such, device 200 may appear, superficially, to be the same as device 100, while in reality it will promote tissue growth in a totally different way upon self-inflation. In addition, device 200 includes bidirectional control of expansion such that the device expands laterally prior to expanding in height. As such, the device is retained within the tissue without prematurely extruding from the tissue.

In one embodiment, the copolymers used in the device 100 or 200 can be extruded into filaments (e.g., 1-2 mm diameter strands) that can be fed into a typical Fused Deposition Modeling (FDM) 3D printer. The 3D printed part is then pressed twice in a hot press, such as between about 150° C. and about 250° C. (temperature will vary based on the exact materials used). The resulting printed part is pressed in one direction, held and cooled, then released; and then pressed again with heat in a direction orthogonal to the first and allowing the material to cool before releasing. The pressing during cooling results in the built-in directionality of the device 100 or 200.

Bone-Growing

As shown in FIGS. 1-4, the unexpanded shape of the devices 100 and 200 is substantially flat, and the expanded shape of the devices 100 and 200 maintains a flat bottom while growing directionally. That is, devices 100 and 200 are design to create displacement in the direction where a palatal defect is expected to be found. The flat or un-displaced sides can be designed to fit with a structure, such as bone, that provides a stable base. That is, the devices 100 and 200 can be designed to fit in a next or mechanical anchor point so that self-inflation creates the desired level of force, rather than simply causing the device itself to shift.

By designing the devices 100 and 200 to nest at an anchor point in this way, the level of stress or strain causes tissue growth where desired. The embodiment(s) contemplated herein describe device geometries in which the base (bone-side) of the device has a cross-sectional area that is equal to or larger than that of the device's apex (mucosa-side).

Design Process

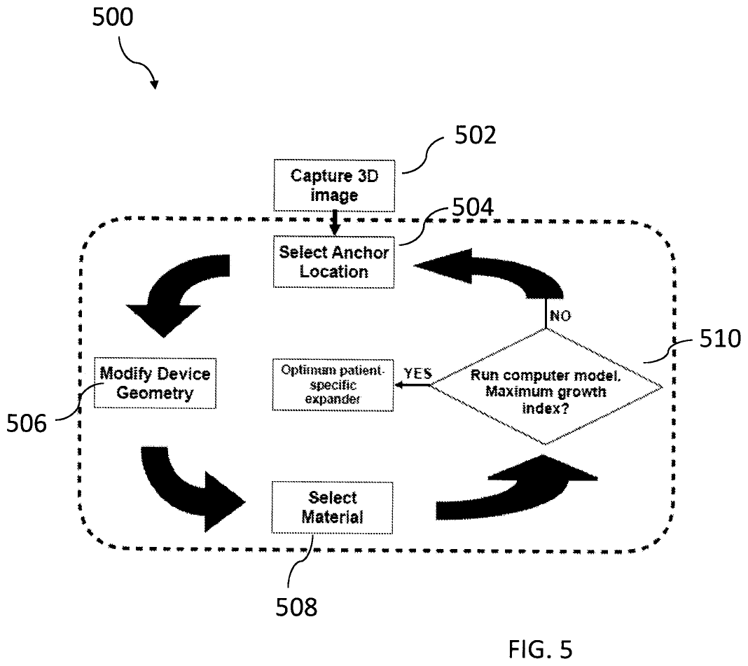

The design of devices described herein, such as device 100 and 200, can be accomplished by following the algorithm describe in FIG. 5.

As shown in FIG. 5, a method (500) for designing a device as described herein can include:
Capturing a three-dimensional image of a palatal defect with tissues adjacent to the defect (at 502);
Selecting an anchor location for the device (at 504);
Specifying an initial device geometry that is compatible with the patient's anatomy and modifying the device geometry as necessary (at 506);
Determining an appropriate material or set of materials and corresponding arrangement within the device to generate tissue growth (at 508);
Running a computer model of implanted device expansion, expected strain and stress at the palatal defect; and expected growth or resorption of soft tissue and bone; where a growth index is computed that describes soft and hard tissue growth (at 510); and
Modifying the anchor location, device geometry, or arrangement of self-inflating materials within the device based upon the model, and repeating iteratively to improve palatal defect repair speed and reduce tissue deficits.

It should be understood that while FIG. 5 refers to “optimum” patient-specific expanders, the method is iterative and may be used to approach an optimum expander for a certain number of loops without actually resulting in a quantitatively optimum expander. Indeed, depending on the desired speed of growth, an “optimum” patient-specific expander is somewhat subjective. In any event, method 500 can be used to iteratively approach a desired growth rate and pattern for a specific patient.

### EXAMPLES

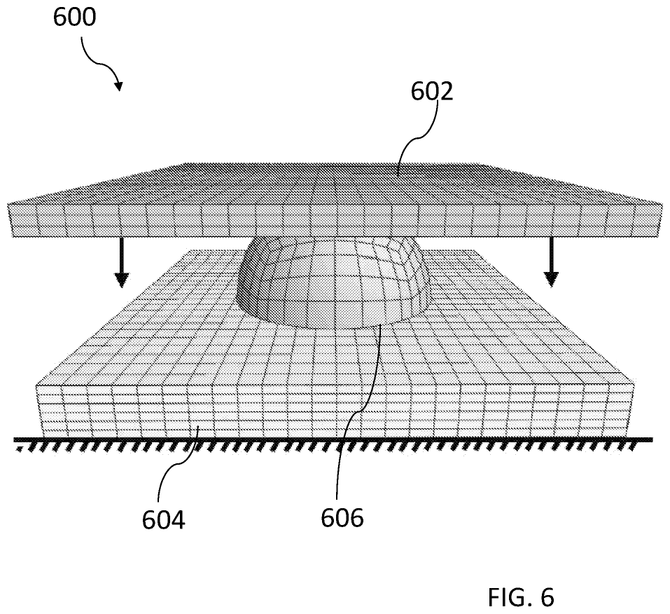

FIG. 6 shows a framework 600 for modeling the shapes of expanders or components thereof and how they initiate mucosal tissue growth. As shown in FIG. 6, mucosal tissue 602 is separated from bone 604 by an expander 606. The framework 600 is shown in exploded view (as depicted by the arrows and in fact the mucosal tissue 602 is flush with the bone 604 such that the expander 606 creates mechanical stress on the mucosal tissue 602 as it stretches over the expander 606.

It has been discovered that some mechanical stress stimulates mucosal tissue growth. In general, growth is stimulated in the range of ˜1 to 1.4 MPa, or more preferably between ˜1 to 1.2 MPa. The examples below show the effects of differently-shaped expanders on force created.

### Example 1—Dome-Shaped Expander

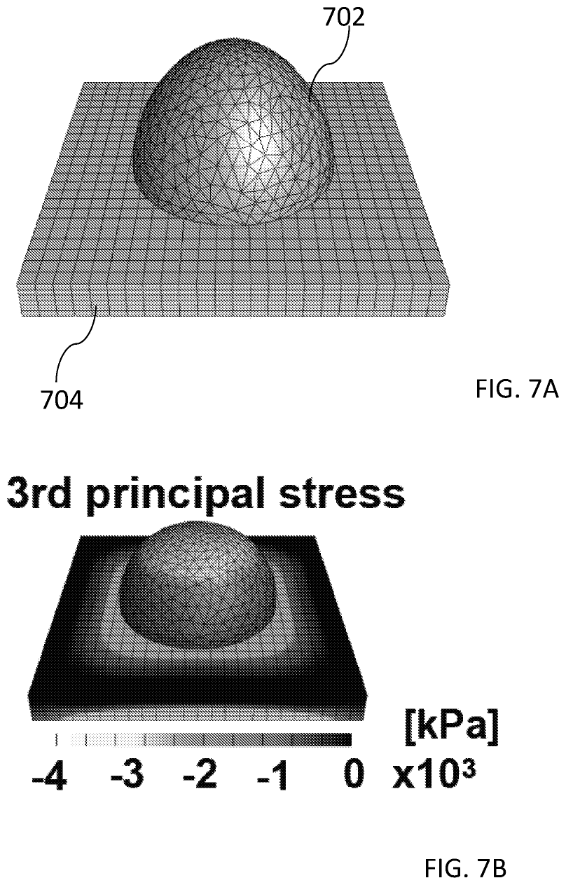

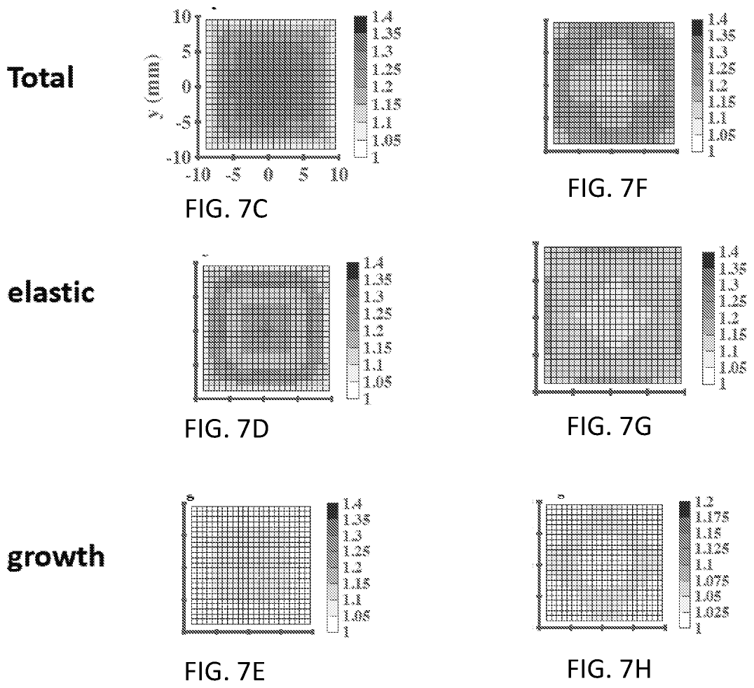

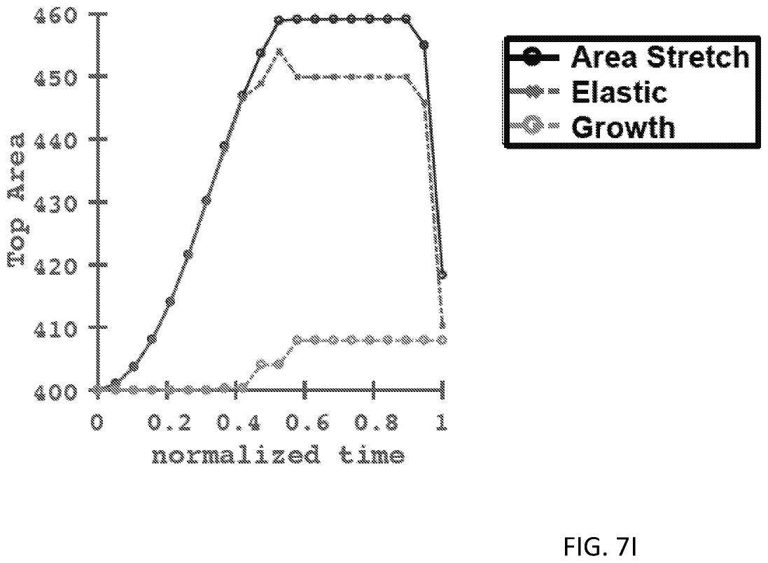

FIGS. 7A-7I show the effect of introducing a dome-shaped expander in the framework of FIG. 6. As shown in FIG. 7A, a dome-shaped expander 706 is arranged on the bone 704 (the mucosal tissue is not shown in this view for clarity). FIG. 7B shows the mechanical stress on the expander 706 and bone 704 due to mechanical interference between the mucosal tissue (not shown, but see 602 of FIG. 6 for reference) and the dome-shaped expander 706.

FIGS. 7C-7E show stress on the top surface of the mucosal tissue (i.e., at a plane spaced slightly away from the expander 706). FIG. 7C shows total mechanical stress applied to the tissue caused by the expander 706. Total stress includes an elastic portion (as shown in FIG. 7D) and is also affected by growth (as shown in FIG. 7E).

Likewise, FIGS. 7F-7H show stress at the bottom surface of the mucosal tissue (i.e., at a plane directly adjacent either the bone 704 or the expander). FIG. 7F shows total stress, while FIG. 7G shows elastic stress and FIG. 7H shows change in stress related to growth of the mucosal tissue.

FIG. 7I shows the relationship between elastic stretch, growth, and total area stretch for the dome-shaped expander 706. As shown in FIGS. 7C-7H, the specific growth rate and stress at any given point varies according to a geometric pattern caused by the geometric shape of the dome. A surgeon wishing to cause tissue growth at a given distance from the bone can use models such as the ones shown herein to determine where stress will be induced and therefore where growth will be promoted.

### Example 2—Rectangular Prism Expander

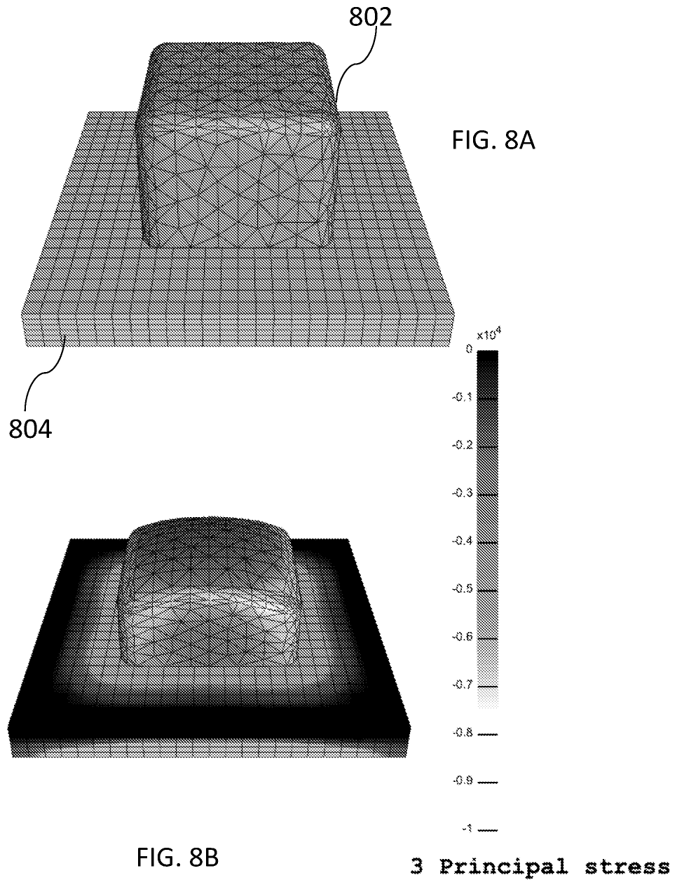

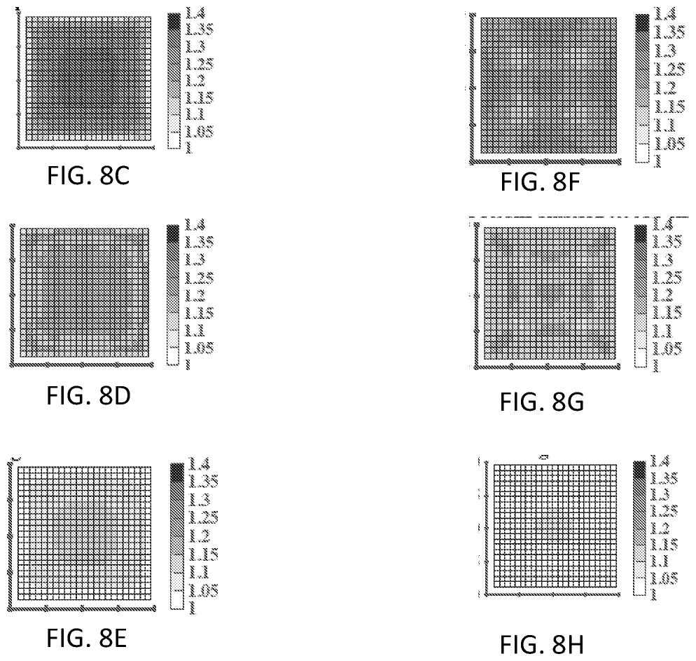

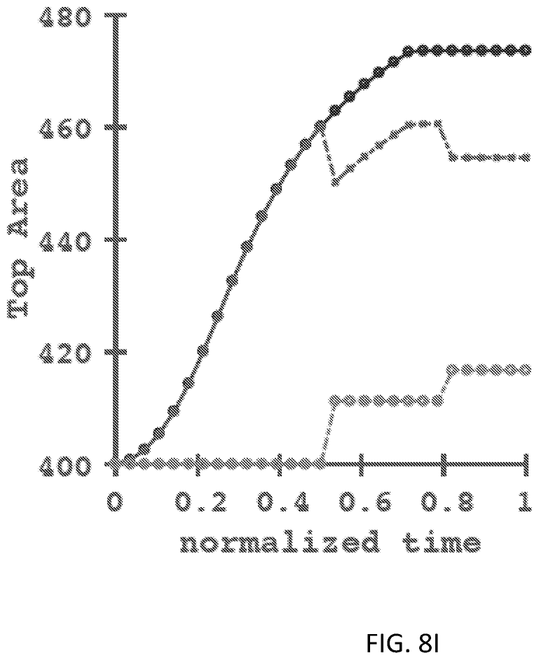

FIGS. 8A-8I show the effect of introducing a rectangular prism-shaped expander in the framework of FIG. 6. As shown in FIG. 8A, a rectangular prism-shaped expander 806 is arranged on the bone 804 (the mucosal tissue is not shown in this view for clarity). FIG. 8B shows the mechanical stress on the expander 806 and bone 804 due to mechanical interference between the mucosal tissue (not shown, but see 602 of FIG. 6 for reference) and the rectangular prism-shaped expander 806.

FIGS. 8C-8E show stress on the top surface of the mucosal tissue (i.e., at a plane spaced slightly away from the expander 806). FIG. 8C shows total mechanical stress applied to the tissue caused by the expander 806. Total stress includes an elastic portion (as shown in FIG. 8D) and is also affected by growth (as shown in FIG. 8E).

Likewise, FIGS. 8F-8H show stress at the bottom surface of the mucosal tissue (i.e., at a plane directly adjacent either the bone 804 or the expander). FIG. 8F shows total stress, while FIG. 8G shows elastic stress and FIG. 8H shows change in stress related to growth of the mucosal tissue.

FIG. 8I shows the relationship between elastic stretch, growth, and total area stretch for the rectangular prism-shaped expander 806. As shown in FIGS. 8C-8H, the specific growth rate and stress at any given point varies according to a geometric pattern caused by the geometric shape of the rectangular prism. A surgeon wishing to cause tissue growth at a given distance from the bone can use models such as the ones shown herein to determine where stress will be induced and therefore where growth will be promoted.

### Example 3—Rectangular Prism Expander

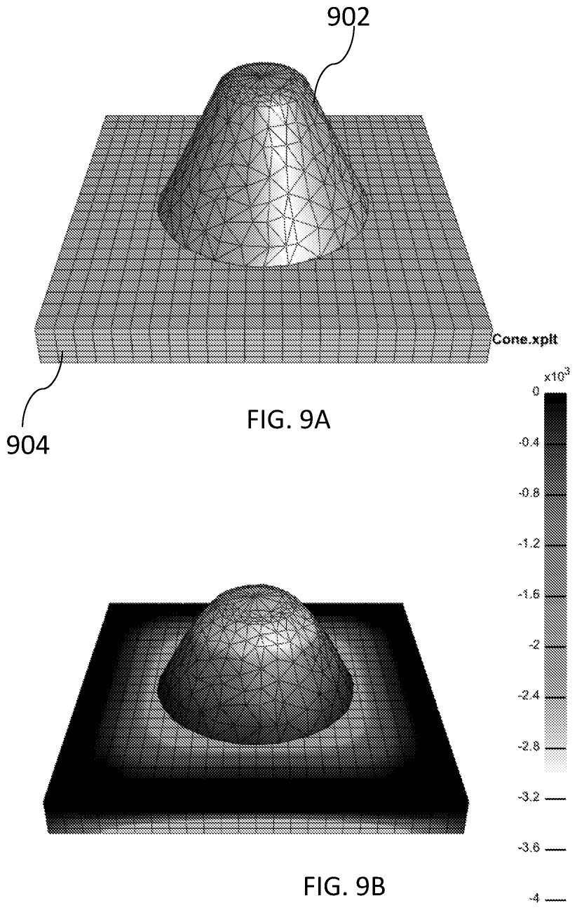

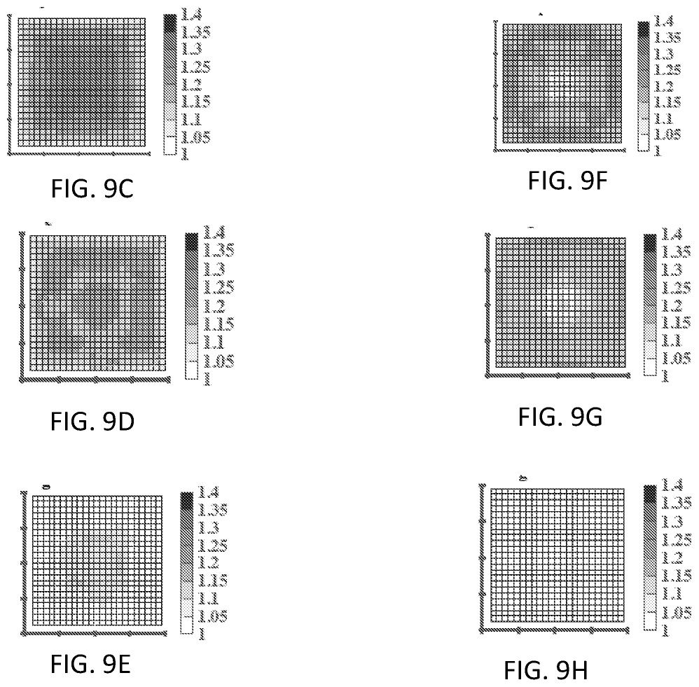

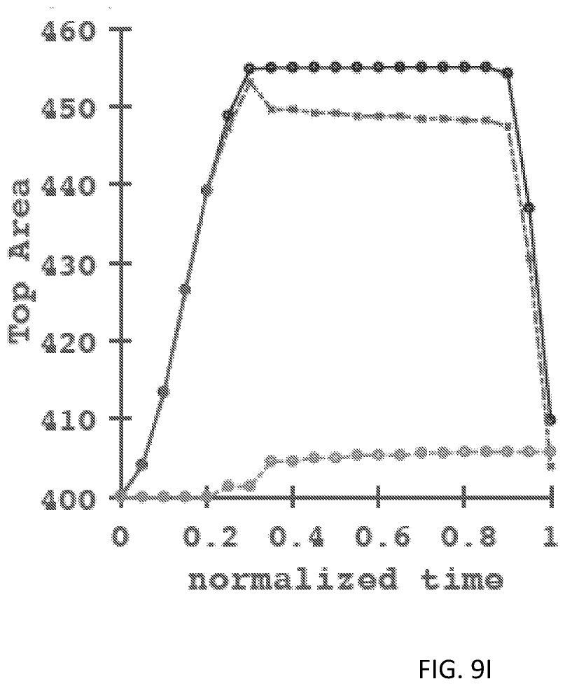

FIGS. 9A-9I show the effect of introducing a cone-shaped expander in the framework of FIG. 6. As shown in FIG. 9A, a cone-shaped expander 906 is arranged on the bone 904 (the mucosal tissue is not shown in this view for clarity). FIG. 9B shows the mechanical stress on the expander 906 and bone 904 due to mechanical interference between the mucosal tissue (not shown, but see 602 of FIG. 6 for reference) and the cone-shaped expander 906.

FIGS. 9C-9E show stress on the top surface of the mucosal tissue (i.e., at a plane spaced slightly away from the expander 906). FIG. 9C shows total mechanical stress applied to the tissue caused by the expander 906. Total stress includes an elastic portion (as shown in FIG. 9D) and is also affected by growth (as shown in FIG. 9E).

Likewise, FIGS. 9F-9H show stress at the bottom surface of the mucosal tissue (i.e., at a plane directly adjacent either the bone 904 or the expander). FIG. 9F shows total stress, while FIG. 9G shows elastic stress and FIG. 9H shows change in stress related to growth of the mucosal tissue.

FIG. 9I shows the relationship between elastic stretch, growth, and total area stretch for the cone-shaped expander 906. As shown in FIGS. 9C-9H, the specific growth rate and stress at any given point varies according to a geometric pattern caused by the geometric shape of the cone. A surgeon wishing to cause tissue growth at a given distance from the bone can use models such as the ones shown herein to determine where stress will be induced and therefore where growth will be promoted.

It should be understood that by combining cones, domes, rectangular prisms, or other shapes, expanders having a desired shape and size to promote desired growth patterns can be generated. Examples of these more complex structures are shown in FIGS. 1-4, for example. Furthermore, choice of material or orientation of sub-structures within the larger expander design can create innumerable different shapes upon expansion to tune the expander device to a particular patient, as described with respect to the method depicted in FIG. 5.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A device for palatal repair comprising:
a plurality of polymeric fibers arranged into a body, the body configured to expand directionally upon exposure to a liquid; and
a coating configured to provide a controlled start time of the device.

2. The device of claim 1, wherein the body comprises a self-inflating body made into a first primary form, the self-inflating body being programmed with thermomechanical conditioning to assume a secondary insertion shape, wherein exposure to interstitial fluid causes the self-inflating body to transition from the secondary insertion shape to the first primary form.

3. The device of claim 2, wherein the polymeric fibers arranged to form the self-inflating body are formed of one or more biocompatible polymers selected from the group comprising:
methylcellulose, poly(acrylamide), poly(caprolactone), poly(ethylene glycol), poly(glycolic acid), poly(glycerol sebacate), poly(2-hydroxyethyl methacrylate), poly(lactic acid), poly(propylene fumerate), poly(vinyl alcohol), poly(vinyl pyrrolidone), poly(methyl methacrylate) and co-polymers thereof.

4. The device of claim 3, wherein one or both of the first primary form and the polymer/copolymer of the self-inflating body is selected to expand directionally in one or more directions selected from height, length and width.

5. The device of claim 2, wherein the polymeric fibers arranged to form the self-inflating body are formed of Poly (vinyl alcohol)-graft polyurethane with resorcinol.

6. The device of claim 2, wherein the self-inflating body is formed such that both the first primary form and the secondary insertion initial shape have a substantially flat bottom to provide a stable base to create a desired level of force.

7. The device of claim 2, wherein the self-inflating body has a device geometry in which a base (bone-side) of the self-inflating body has a cross-sectional area that is equal to or larger than that of an apex (mucosa-side) of the self-inflating body.

8. The device of claim 2, wherein the self-inflating body is formed by 4-D printing that accounts for an initial shape and size at a first implantation time as well as an expected shape and size over a healing period following implantation.

9. The device of claim 8, wherein the healing period is between about 1 day to about 60 days.

10. The device of claim 8, wherein the self-inflating body applies sustained pressure to a tissue area during the healing period to achieve an elastic area stretch ratio of between 1.1 and 2.0.

11. The device of claim 10, wherein the elastic area stretch ratio is between 1.1 and 1.5.

12. The device of claim 2, wherein the first primary form is formed of shapes selected from cones, domes, rectangular prisms and combinations thereof.

13. The device of claim 2, wherein the first primary form is configured to subject mucosal tissue to mechanical stress when the self-inflating body is positioned to separate mucosal tissue and bone, said mechanical stress being within a range of about 1.0 to about 1.4 MPa.

14. The device of claim 13, wherein the mechanical stress is within the range of about 1.0 to about 1.2 Mpa.

15. The device of claim 1, wherein the coating comprises a biocompatible coating selected to break down in response to contact with interstitial fluid over a desired time period.

16. The device of claim 15, wherein the biocompatible coating is selected from poly(ethylene glycol), silicones and poly(ethylene glycol) diacrylate.

17. The device of claim 16, wherein the biocompatible coating comprises biocompatible materials selected from initiators, plasticizers, fillers and crosslinkers.

18. A method for designing a palatal repair device comprising:
capturing a three-dimensional image of a palatal defect with tissues adjacent to the palatal defect;
selecting an anchor location for the palatal repair device;
specifying an initial device geometry that is compatible with an anatomy adjacent the palatal defect;
determining an appropriate material or set of materials and corresponding arrangement within the device to generate tissue growth;
modeling implanted device expansion, expected strain and stress at the palatal defect; and
expected growth or resorption of soft tissue and bone, wherein a growth index is computed that describes soft and hard tissue growth; and
modifying the anchor location, device geometry, or arrangement of self-inflating materials within the device based upon the model.

19. The method of claim 18, wherein the steps of modifying is iteratively repeated to improve palatal defect repair speed and reduce tissue deficits.

20. The method of claim 19, wherein the iteratively repeated step of modifying is based upon a desired growth rate and pattern for a specific patient.
